(19) United States
(12) Patent Application Publication
(10) Pub. No.: US 2020/0253917 A1
(43) Pub. Date: Aug. 13, 2020

(54) COMPOSITIONS AND METHODS FOR MODULATING HAIR GROWTH

(71) Applicant: The Regents of the University of California, Oakland, CA (US)

(72) Inventors: William E. Lowry, Los Angeles, CA (US); Heather R. Christofk, Los Angeles, CA (US); Matilde Miranda, Los Angeles, CA (US); Aimee Flores, Los Angeles, CA (US)

(21) Appl. No.: 16/651,835

(22) PCT Filed: Sep. 28, 2018
(86) PCT No.: PCT/US2018/053351

§ 371 Date: Mar. 27, 2020

Related U.S. Application Data
(60) Provisional application No. 62/566,031, filed on Sep. 29, 2017.

(51) Int. Cl. A61K 31/352; A61K 45/06; A61K 31/155; A61K 31/357; A61P 17/14

(52) U.S. Cl. CPC A61K 31/352; A61K 45/06; A61P 17/14; A61K 31/357; A61K 31/155

(57) ABSTRACT

The present disclosure relates to pharmaceutical compositions containing electron transport chain (ETC) inhibitors, which are capable of promoting hair growth. The disclosure further relates to methods of promoting hair growth or treating conditions or disorders affecting hair growth, such as baldness or alopecia.

### RELATED APPLICATIONS

This application claims the benefit of U.S. Provisional Patent Application No. 62/566,031, filed on Sep. 29, 2017. The contents of this application are hereby incorporated by reference in their entirety.

### BACKGROUND

Hair follicle stem cells (HFSCs) undergo successive rounds of quiescence (telogen) punctuated by brief periods of proliferation correlating with the start of the hair cycle (telogen-anagen transition). Proliferation or activation of HFSCs is well known to be a prerequisite for advancement of the hair cycle. Despite advances in treatment options, baldness and alopecia continue to be conditions that cannot be successfully treated in all individuals. Some of the existing treatments are inconvenient for users, others require surgical intervention or other invasive procedures. Additional therapies are needed.

### SUMMARY OF THE INVENTION

In certain aspects, the present disclosure provides pharmaceutical compositions comprising inhibitors of the Electron Transport Chain (ETC). In certain embodiments, the pharmaceutical compositions are formulated for topical administration.

In certain aspects, the present disclosure provides methods of promoting hair growth, comprising administering to a patient a therapeutically effective amount of a composition as described herein.

### BRIEF DESCRIPTION OF THE DRAWINGS

FIGS. 1A-1D show that topical treatment with ETC inhibitor can promote the hair cycle. FIG. 1A: Mice were shaved at day 50 (Telogen) and topically treated with Phenformin (5 uM) every other day for 2-3 weeks. The image demonstrates new pigmentation and then hair growth in response to treatment with Phenformin at 10 and 16 days, and H and E staining confirms advancement of the hair cycle (BOTTOM). FIG. 1B: Quantification of changes to the hair cycle in treatment and control. Quantification performed across 13 vehicle and 9 Phenformin treated male mice. FIG. 1C: Changes to the thickness of the epidermis, dermis and hypodermis were assessed microscopically and quantified. FIG. 1D: Immunohistochemistry for a marker of HFSCs (Sox9) and proliferation (Ki-67) demonstrated that HFSCs became activated in response to ETC inhibition by Phenformin, Rotenone and Antimycin A. Scale bars for A and C indicate 50 micrometers. Scale bars for D indicate 25 micrometers.

FIGS. 2A and 2B show that topical ETC inhibition increases lactate production. FIG. 2A: Mice were treated topically with the indicated ETC inhibitor for 48 hours. Total epidermis was isolated, lysed and subjected to LDH activity assay. Relative LDH activity is presented as the rate of activity over 30 min in two different animals. FIG. 2B: Mice were treated topically with ETC inhibitor for 48 hours (TOP) or 10 days (BOTTOM). Total epidermis was isolated, and metabolites were extracted and subjected to metabolomics. Heatmap indicates relative levels of metabolites related to glycolysis and the TCA cycle.

FIGS. 3A-3D show that topical treatment with ETC inhibitor can promote the hair cycle. FIG. 3A: Mice were shaved at day 50 (Telogen) and topically treated with antimycin A or Rotenone every other day for 2-3 weeks. The image demonstrates new pigmentation in response to treatment with either Rotenone or Antimycin A, and H and E staining confirms advancement of the hair cycle (BOTTOM). FIG. 3B: Quantification of changes to the hair cycle in treatment and control. Quantification was performed with 13 vehicle treated, 11 Rotenone treated, and 9 Antimycin A treated male animals. FIG. 3C: Changes to the thickness of the epidermis, dermis and hypodermis were assessed microscopically and quantified. FIG. 3D: Immunolocalization was performed to detect evidence of inflammation due to topical application of ETC inhibitors. Both vehicle and ETC inhibitor treated skin was immunostained for phosphor-EGFR (chemokine receptor), CD11b (marker of macrophages), and IL6 (Chemokine). Vehicle treated skin from a wounded animal with hyperplastic epidermis was used as a positive control for markers of inflammation. Scale bars indicate 50 micrometers.

FIGS. 4A and 4B. Treatment with ETC inhibitors can accelerate the hair cycle in aged mice. FIG. 4A: Female mice were shaved at 17 months of age and then treated with vehicle or the indicated ETC inhibitor every other day for up to 30 days. Images taken over time indicate the ETC inhibition promotes a more complete re-growth of hair after shaving in aged mice. Quantification of phenotype emergence from two pairs of animals are presented on the right. Data shown are representative of three independent experiments with 10 mice each. FIG. 4B, Metabolites were isolated from sorted HFSCs from skin treated with ETC inhibitors at the end of the hair cycle experiment depicted in A. Heatmap shows relative levels of the indicated metabolites.

### DETAILED DESCRIPTION OF THE INVENTION

While many signaling pathways have been implicated in control of activation of adult hair follicle stem cells (HFSCs) and the hair cycle, less is known about cell intrinsic mechanisms of stem cell control. Lactate production has been identified as a key cell intrinsic regulator of hair follicle stem cell activity, suggesting that cellular metabolism is important in stem cell activation. Transgenic methods have been used to suggest that transgenic blockade of the Electron Transport Chain (ETC) leads to degeneration of the hair follicle. However, the present disclosure provides composition and methods by which pharmacological abrogation of ETC activity, as opposed to complete ablation of ETC, can promote hair cycle activation without significant cell toxicity. Furthermore, the metabolic data provided herein suggest that ETC inhibition leads to increased pyruvate accessibility for the Ldh enzyme and therefore increased lactate production, which can promote hair cycle activation. Finally, this type of ETC inhibition can even be used to accelerate the hair cycle in aged mice. These results point toward an unexpected and safe method to promote hair follicle stem cell activation.

Over the last three decades, a number of signaling pathways have been identified that act on HFSCs to promote both quiescence as well as their activation. With respect to intrinsic mechanisms of HFSC regulation, less is known about the cellular metabolism of individual cell types in the epidermis. In general, it has been presumed that somatic cells use mostly the electron transport chain (ETC) to produce energy from pyruvate that was generated by the uptake and processing of glucose, while early embryonic and cancer cells are thought to also rely on production of lactate from pyruvate. HFSCs balance the production of energy through the ETC with the production of lactate as well. Previous efforts to define metabolic activities in the epidermis focused on measurements of enzyme activities on entire follicles. In addition, several studies used transgenic models targeting the entire epidermis (including the follicle) for deletion of ETC components. Those studies suggested that genetic blockade of the ETC leads to degeneration of the follicle. However, it is not clear whether inhibition of ETC complexes—as opposed to genetic ablation of ETC complexes—would affect cell metabolism or fate decisions.

The present disclosure shows that inhibiting ETC activity causes proliferation of HFSCs and promotes hair growth. As used herein, the term “ETC inhibitor” includes any agent that is capable of inhibiting ETC complexes I, II, III, or IV, preferably ETC complexes I or III. Inhibitors of each of these complexes are known in the art. Inhibitors of ETC complex I include metformin, phenformin, buformin, rotenone, epiberberine, piericidin A, amytal, capsaicin, haloperidol, risperidone, bupivacaine, lidocaine, halothane, dantrolene, phenyloin, clofibrate, and fenofibrat. Inhibitors of ETC complex II include sodium malonate, thenoyltrifluoroacetone, cyclophosphamide, and ketoconazole. Inhibitors of ETC complex III include antimycin A, acetaminophen, isoflurane, and sevoflurane. Inhibitors of ETC complex IV include cephaloridine, cefazolin, and cefalotin. Certain ETC inhibitors are generally described in U.S. Pat. No. 8,993,587, which is hereby incorporated by reference as if fully set forth herein.

In certain aspects, the present disclosure provides pharmaceutical compositions formulated for topical administration comprising inhibitors of the Electron Transport Chain (ETC). As described herein, ETC inhibitors cause proliferation of HFSCs and can thereby promote hair growth.

In certain embodiments, the electron transport chain inhibitor is an inhibitor of electron transport chain complex I, II, III, or IV. In certain embodiments, the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, sodium malonate, thenoyltrifluoroacetone, cyclophosphamide, ketoconazole, antimycin A, acetaminophen, isoflurane, sevoflurane, cephaloridine, cefazolin, or cefalotin; or a pharmaceutically acceptable salt thereof.

In certain embodiments, the electron transport chain inhibitor is an inhibitor of electron transport chain complex I or III. In certain embodiments, the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, antimycin A, acetaminophen, isoflurane, or sevoflurane. In certain embodiments, the electron transport chain inhibitor is rotenone, phenformin, or antimycin A.

In certain aspects, the present disclosure provides methods of promoting hair growth, comprising administering to a patient a therapeutically effective amount of a composition comprising an ETC inhibitor as described herein. In certain embodiments, the condition or disorder is baldness or alopecia.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound as disclosed herein and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound as disclosed herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound as disclosed herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound as disclosed herein, with the carrier and, optionally, one or more accessory ingredients. In general, the formulations are prepared by uniformly and intimately bringing into association a compound as disclosed herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as disclosed herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound as disclosed herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound as disclosed herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of the agents disclosed herein in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

### Examples

The invention now being generally described, it will be more readily understood by reference to the following examples which are included merely for purposes of illustration of certain aspects and embodiments of the present invention, and are not intended to limit the invention.

### Example 1: Effect of ETC Activity on HFSC Activation

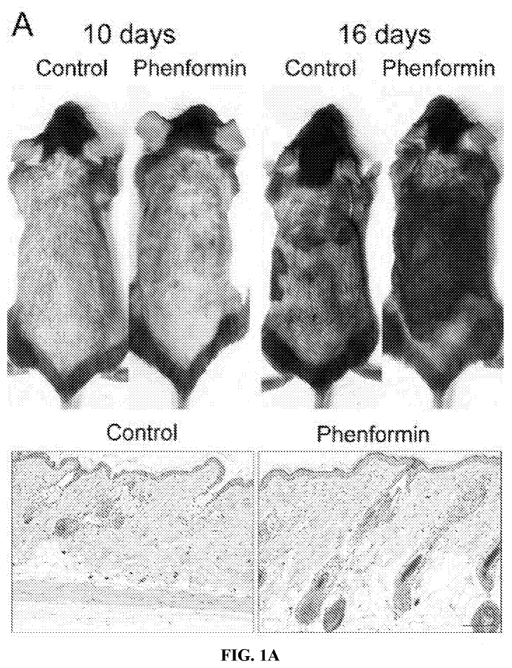

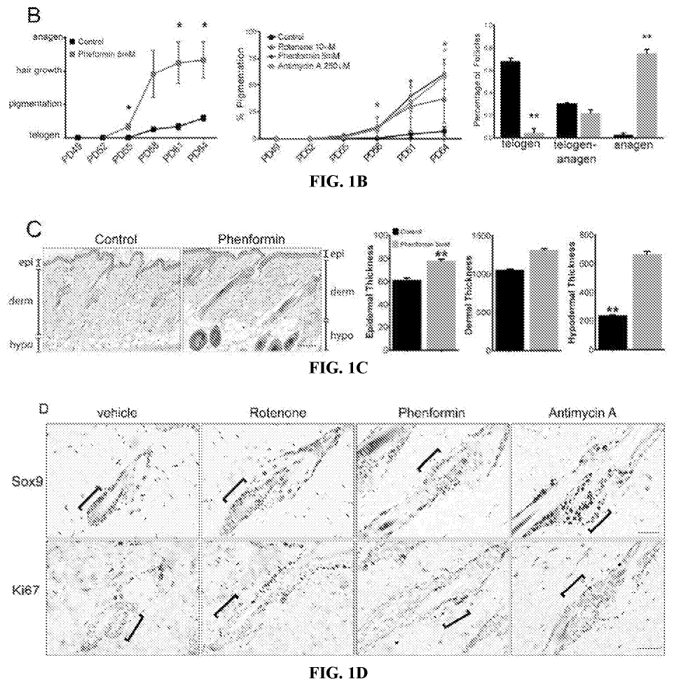

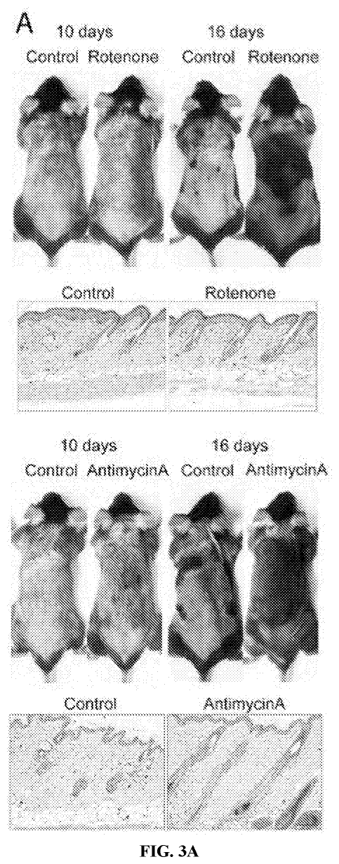

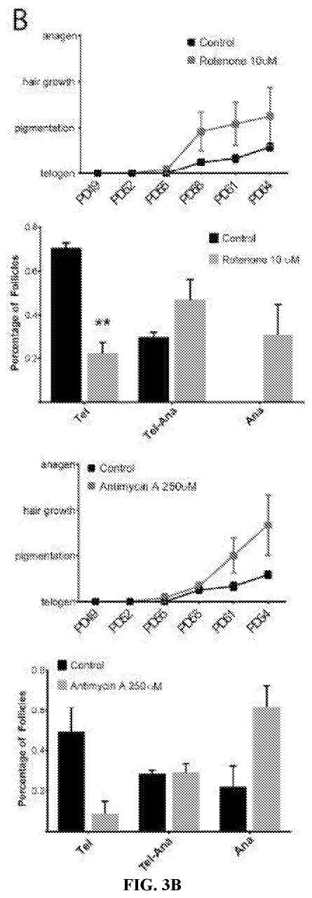

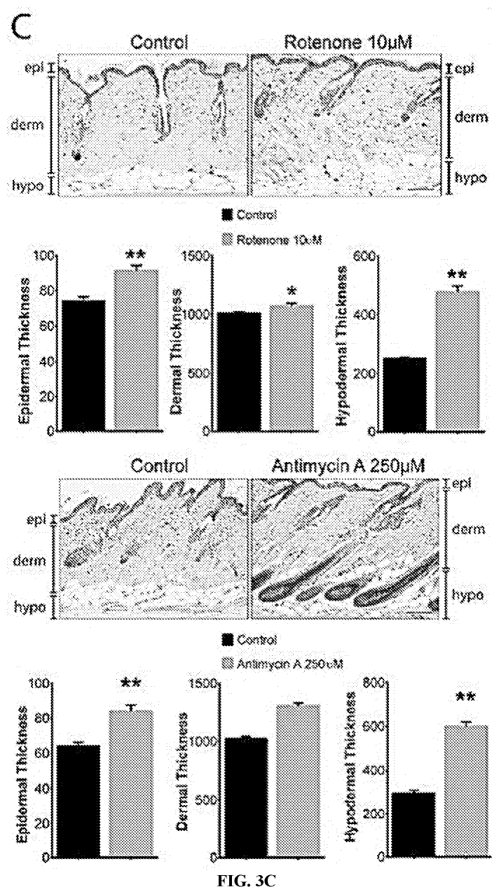

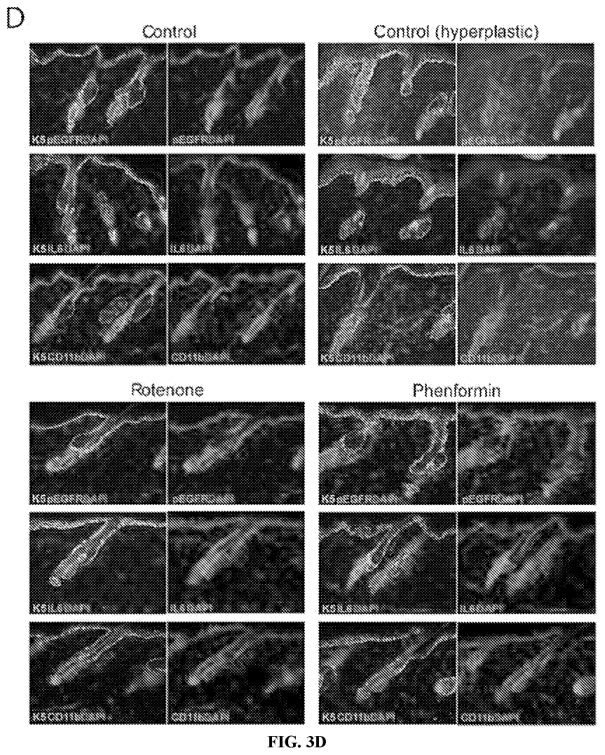

To determine whether manipulation of ETC activity could affect HFSC activation, various inhibitors of ETC components were topically applied to mice during a resting phase of the hair cycle. The topical formulation was prepared by suspending the active ingredient in PLO Ultramax Gel (lecithin organogel). At postnatal day 50, the hair follicle is in telogen, a resting phase where the stem cells of the follicle are quiescent until the start of the next hair cycle at day 70-80. Rotenone, Phenformin, and Antimycin A are established inhibitors of Complex I, and Complex III, respectively. Animals were shaved at postnatal day 47 and treated with the indicated compounds or vehicle on the shaved area every 48 hours for the indicated duration. After 3-4 treatments (8-12 days), animals treated with ETC inhibitors began to show signs of hair cycle activation macroscopically, judged by pigmentation of the skin in black mice whereas vehicle treated mice did not show significant pigmentation for at least 20 days (FIGS. 1A and 3A). The epidermis of murine skin becomes pigmented upon induction of the hair cycle, which is indicative of the generation of melanocytes injecting pigment (melanin) into the keratinocytes that go on to make the hair shaft, as well as those in the interfollicular epidermis. Therefore, the induction of pigmentation observed after 8-12 days in ETC inhibitor treated mice was most likely indicative of hair cycle activation induced by this treatment.

### Example 2: Pathology of ETC-Inhibited Tissues

To demonstrate that the pigmentation induced by ETC inhibition was in fact due to changes in hair follicle stem cell activation, tissue was harvested and subjected to pathology. Histological analysis showed that follicles in backskin treated with ETC inhibitors promoted a normal telogen-to-anagen transition (FIGS. 1B and 3B). These findings were also in stark contrast to previous studies showing that transgenic abrogation of the ETC led to hair follicle degeneration.

### Example 3: Skin Thickness Measurements

To determine whether the hair cycle induction driven by ETC inhibition was typical, the thickness of each layer of skin was measured at different stages of treatment. As shown in FIG. 1C, all of the ETC inhibitors increased the thickness of the epidermis, dermis, and particularly the hypodermis, suggesting a strong expansion of the adipocytes. Analysis of ETC inhibited skin showed a strong increase in Ki67 in HFSCs a week after treatment, evidence of HFSC activation in response to ETC inhibition (FIGS. 1D and 3D). To determine whether application of the ETC inhibitors promoted inflammation, which could cloud interpretation of hair cycle data, various markers of chemokine response and the presence of inflammatory immune cells were assessed after treatment. There was no evidence of significant inflammation by these measures in response to ETC inhibition (FIG. 3D).

### Example 4: Metabolic Measurements

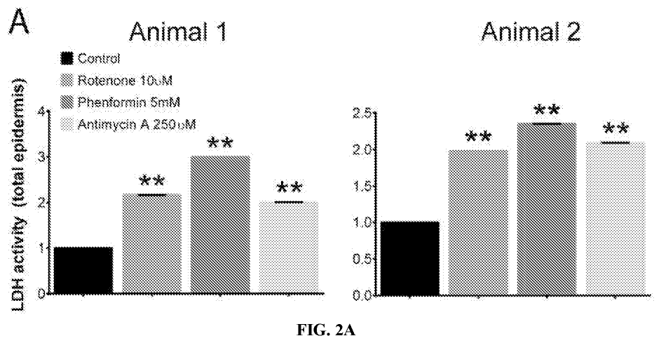

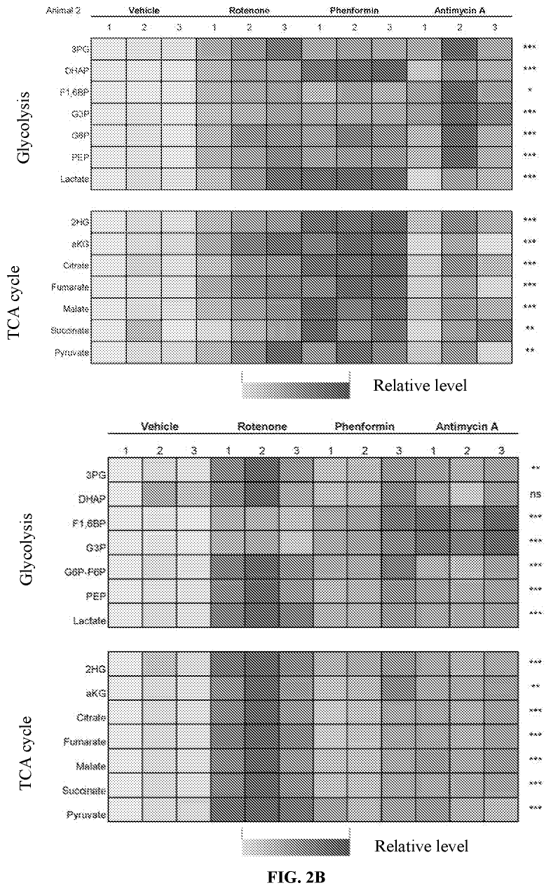

To determine the effect on cellular metabolism of ETC inhibition by Rotenone, Phenformin and Antimycin A, two measures of metabolic pathways were performed. First, LDH activity was quantified on cells isolated from the epidermis treated with ETC inhibitors for 48 hours (FIG. 2A). Next, metabolomics was employed on sorted HFSCs with and without treatment for either 48 hours or 10 days. These analyses indicated an increase in lactate levels as well as several other glycolytic intermediates in response to ETC inhibition by Rotenone, Phenformin and Antimycin A (FIG. 2B).

### Example 5: Effect of ETC Inhibition on Aged Mice

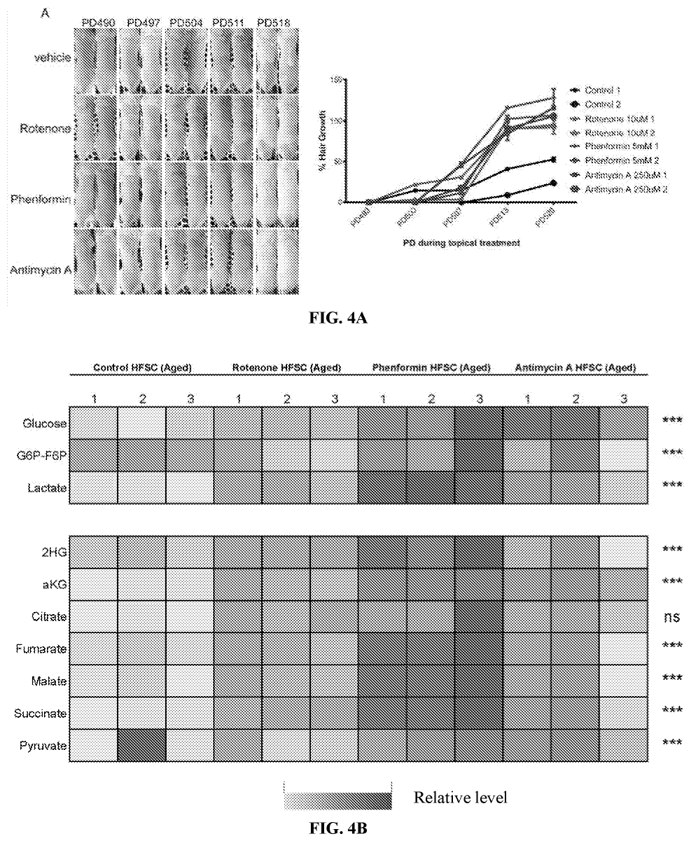

As mice age, the hair cycle is known to become protracted such that upon shaving, only portions of the backskin show regrowth of hair within a 1-2 months. Various batches of aged mice (at least 17 months) were treated for 30 days with ETC inhibitors to determine whether this metabolic manipulation could stimulate the hair cycle even in dormant follicles. Topical application of Phenformin, Rotenone or Antimycin A all led to more complete hair regrowth across the entire backskin on a similar time course to that of younger mice (FIG. 4A). As in younger animals, treatment with these ETC inhibitors led to an increase in lactate pool levels as measured by metabolomics (FIG. 4B).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A pharmaceutical composition comprising an electron transport chain inhibitor and a pharmaceutically acceptable excipient, wherein the pharmaceutical composition is formulated for topical administration.

2. The pharmaceutical composition of claim 1, wherein the electron transport chain inhibitor is an inhibitor of electron transport chain complex I, II, III, or IV.

3. The pharmaceutical composition of claim 1, wherein the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, sodium malonate, thenoyltrifluoroacetone, cyclophosphamide, ketoconazole, antimycin A, acetaminophen, isoflurane, sevoflurane, cephaloridine, cefazolin, or cefalotin; or a pharmaceutically acceptable salt thereof.

4. The pharmaceutical composition of claim 2, wherein the electron transport chain inhibitor is an inhibitor of electron transport chain complex I or III.

5. The pharmaceutical composition of claim 4, wherein the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, antimycin A, acetaminophen, isoflurane, or sevoflurane.

6. The pharmaceutical composition of claim 5, wherein the electron transport chain inhibitor is rotenone, phenformin, or antimycin A.

7. A method of promoting hair growth, comprising administering to a patient a therapeutically effective amount of a composition of claim 1.

8. A method of treating a condition or disorder affecting hair growth, comprising administering to a patient a therapeutically effective amount of a composition of any claim 1.

9. The method of claim 8, wherein the condition or disorder is baldness or alopecia.

10. The method of claim 7, wherein the electron transport chain inhibitor is an inhibitor of electron transport chain complex I, II, III, or IV.

11. The method of claim 7, wherein the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, sodium malonate, thenoyltrifluoroacetone, cyclophosphamide, ketoconazole, antimycin A, acetaminophen, isoflurane, sevoflurane, cephaloridine, cefazolin, or cefalotin; or a pharmaceutically acceptable salt thereof.

12. The method of claim 10, wherein the electron transport chain inhibitor is an inhibitor of electron transport chain complex I or III.

13. The method of claim 12, wherein the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, antimycin A, acetaminophen, isoflurane, or sevoflurane.

14. The method of claim 13, wherein the electron transport chain inhibitor is rotenone, phenformin, or antimycin A.

15. The method of claim 8, wherein the electron transport chain inhibitor is an inhibitor of electron transport chain complex I, II, III, or IV.

16. The method of claim 8, wherein the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, sodium malonate, thenoyltrifluoroacetone, cyclophosphamide, ketoconazole, antimycin A, acetaminophen, isoflurane, sevoflurane, cephaloridine, cefazolin, or cefalotin; or a pharmaceutically acceptable salt thereof.

17. The method of claim 16, wherein the electron transport chain inhibitor is an inhibitor of electron transport chain complex I or III.

18. The method of claim 17, wherein the electron transport chain inhibitor is metformin, phenformin, buformin, rotenone, epiberberine, piericidin A, amytal, capsaicin, haloperidol, risperidone, bupivacaine, lidocaine, halothane, dantrolene, phenyloin, clofibrate, fenofibrat, antimycin A, acetaminophen, isoflurane, or sevoflurane.

19. The method of claim 18, wherein the electron transport chain inhibitor is rotenone, phenformin, or antimycin A.
